(19) United States
(12) Patent Application Publication
(10) Pub. No.: US 2011/0046444 A1
(43) Pub. Date: Feb. 24, 2011

(54) CAVITY EXAMINATION DEVICE

(75) Inventors: Uwe Rass, Nürnberg (DE); Joseph Sauer, Strullendorf (DE)

Correspondence Address: LERNER GREENBERG STEMER LLP, P O BOX 2480, HOLLYWOOD, FL, 33022-2480, US

(73) Assignee: SIEMENS MEDICAL INSTRUMENTS PTE. LTD., Singapore (SG)

(21) Appl. No.: 12/863,329

(22) PCT Filed: Jan. 13, 2009
(86) PCT No.: PCT/EP09/50303

§ 371 Date: Jul. 16, 2010

(30) Foreign Application Priority Data
Jan. 18, 2008 (DE) 10 2008 005 070.9
Jan. 18, 2008 (US) 61021958

(51) Int. Cl. A61B 1/00

(52) U.S. Cl. 600/122

(57) ABSTRACT

A cavity examination device has an optical receiving element, such as a lens or a camera, and enables insertion into the cavity, in which the reception element does not touch the cavity wall. The cavity examination device has an optical receiving element which receives light emitted or reflected by a wall of a cavity and a screen made of a flexible material. The screen has a first opening and a second opening. The cross section of the second opening is larger here than the cross section of the first opening, and the second opening is selected with regard to the shape and dimension such that, predominantly, it resiliently rests against the wall of the cavity. The screen is attached by the first opening to a body of the cavity examination device on a side of the cavity examination device, which is inserted first into the cavity. During insertion of the cavity examination device into the cavity, the screen is folded in the direction of the cavity examination device, and as the cavity examination device is moved out of the cavity, it folds over so that the screen is folded away from the cavity examination device as the cavity examination device is removed from the cavity.

The present invention relates to an improved cavity examination device. In particular, the invention relates to a cavity examination device which has an optical reception element, for example a lens, an image sensor or a camera, and allows insertion into the cavity, without the reception element touching the cavity wall.

A wide variety of devices for examining cavities are known. In the field of otorhinolaryngology, for example, a so-called otoscope is used which has a funnel- or cone-shaped tip so that, when examining the cavity (here: auditory canal or nasal opening), the cavity wall (here: auditory canal wall or nasal wall) is not injured and at the same time an optical reception element (here: lens) is guided so that it does not touch the cavity wall. Besides the conventional otoscopes which a doctor uses in order to look directly into the ear or nasal cavity, for example U.S. Pat. No. 5,363,839 discloses a video otoscope likewise with a funnel- or cone-shaped tip, in which a camera records images of the subject of the examination.

Besides otoscopes, which primarily make it possible to examine objects (for example eardrum) lying at the end of the cavity, devices are known which allow three-dimensional measurement of cavities. For example, EP 1 797 813 A1 describes such a device which is also suitable in particular for three-dimensional measurement of the auditory canal, for instance in order to allow optimal adaptation of hearing aids worn in the ear (so-called in-ear hearing aids) to the ear.

The device known from EP 1 797 813 A1, however, is relatively complex. It would be more economical to introduce a comparatively simply constructed pin-shaped device with an integrated camera into the cavity, for example the auditory canal, and analyze the image sequence recorded during insertion and/or removal, and to calculate a 3D model of the cavity from this.

Such a pin-shaped device, however, has disadvantages. On the one hand, contact of the device with the cavity wall, for example the auditory canal, cannot reliably be avoided so that contamination of the front optical element or injury to the auditory canal cannot reliably be prevented. On the other hand flexible obstacles, for instance hairs in the auditory canal, must elaborately be removed before the examination so that they do not interfere with the image sequence being recorded.

It is therefore an object of the present invention to provide an improved cavity examination device, in particular a cavity examination device which has an optical reception element, for example a lens or a camera, and allows insertion into the cavity without the reception element touching the cavity wall.

This object is achieved by a cavity examination device, which comprises the following:
an optical reception element which receives light emitted or reflected by a wall of a cavity; and
a screen made of flexible material,
   which has a first opening and a second opening, the cross section of the second opening being larger than the cross section of the first opening, and the second opening being selected in respect of configuration and dimension so that it predominantly bears resiliently on the wall of the cavity,
   which is fastened with the first opening on a body of the cavity examination device on a side of the cavity examination device inserted first into the cavity, and
   which is folded in the direction of the cavity examination device when the cavity examination device is inserted into the cavity and is folded back when the cavity examination device is moved out of the cavity, so that the screen is folded away from the cavity examination device when the cavity examination device is removed from the cavity.

A screen made of silicone is preferably used, which preferably has essentially the shape of a frustum of a body of rotation (cone frustum, frustum of a paraboloid of rotation).

In a preferred configuration, the screen is dimensioned so that the screen does not interfere with the imaging by the optical reception element, that is to say the screen edge always lies outside the cavity examination device's imaging region defined by the aperture angle of the reception element.

Preferably, the screen can be replaced by screens with a differently configured and/or dimensioned second opening, in order to achieve adaptation of the cavity examination device to different cavities.

For use in tapering cavities, the screen may be slit.

A particularly simple design is achieved when the cavity examination device has a cylindrical body, that is to say the cavity examination device consists for example of a pin-shaped camera with a screen fitted on it.

Such a cavity examination device with a camera is then suitable in particular for recording an image sequence, which is fed to a control device that computes a 3D model of the cavity from the image sequence. The image recording is preferably carried out when removing the cavity examination device from the cavity, since the screen folded away from the cavity examination device then on the other hand provides centering of the camera or the front camera lens and, on the other hand, keeps flexible obstacles on the wall of the cavity (for instance hairs in the auditory canal) away from the image region.

Exemplary embodiments of the invention will be explained in more detail below with the aid of drawings, in which:

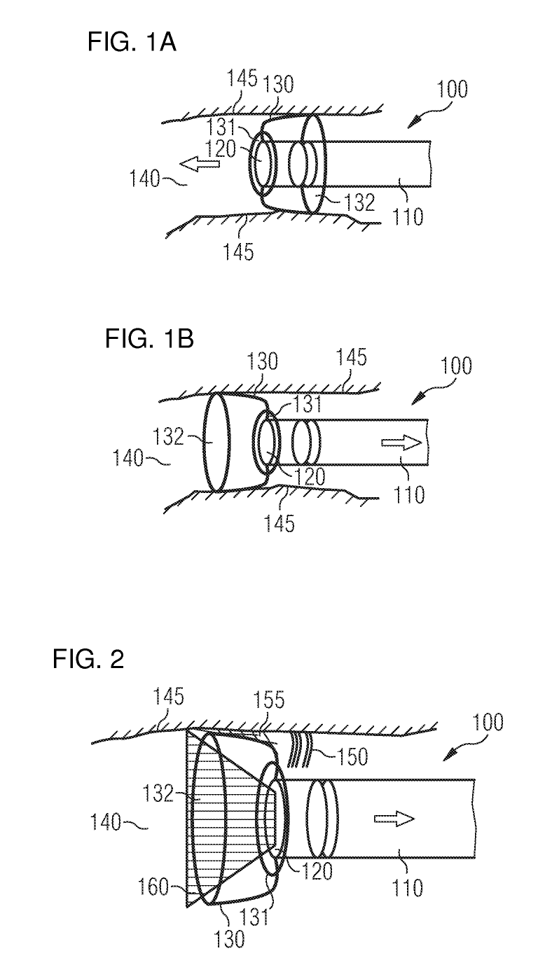

FIG. 1a shows a cavity examination device according to the invention during insertion into a cavity represented in section;

FIG. 1b shows a cavity examination device according to the invention during removal from a cavity represented in section; and

FIG. 2 shows a cavity examination device according to the invention during removal from a cavity represented in section with further details.

FIG. 1 shows a cavity examination device 100 according to the invention, which has a device body 110, and optical reception element 120 and a screen 130. The device 100 is partially inserted into a cavity 140, which is represented in section and has walls 145. The cavity 140 may for example be a human auditory canal, which is bounded by the auditory canal walls. The optical reception element 120 receives light emitted or reflected by the wall 145 of the cavity 140 and processes it either directly, if the reception element 120 is an image sensor, or forwards it via further optical elements, for example to an observer's eye or to an image sensor of a camera.

The cavity 140 is open on at least one side; in the representation of FIG. 1, this is the side through which the cavity examination device 100 is partially inserted into the cavity 140.

In many cases, the cavity 140 is closed on the other side; in the case of the human ear, for example, this is done by the body interior and/or the eardrum. In order to be able to receive light using the reception element 120 in such cases, it is often necessary to feed light into the cavity by means of the cavity examination device 100. Corresponding light sources (not shown) and their integration into the cavity examination device are well known in the art, and will not therefore be discussed separately here.

The screen 130 consists of an elastic material, for example silicone, and is preferably formed as a frustum of a body of rotation, for example as a frustum of a cone or a paraboloid of rotation, although for special applications it may also be a pyramid frustum or any desired hollow body. What is important is merely that the screen 130 has two openings 131, 132, the first opening 131 being smaller than the second opening 132. The first opening 131 is furthermore adapted in respect of configuration and dimension to the body 110 of the cavity examination device 100, so that it can be fastened to it. In the case of a cylindrical body 110, the first opening 131 will approximately be a circle of suitable diameter; in the case of a body 110 with a rectangular cross section, on the other hand, it will be a rectangle with suitable edge lengths.

The second opening 132, on the other hand, is adapted in configuration and dimension to the cavity 140 to be examined, and specifically so that resilient bearing along the contour of the cavity wall 145 is predominantly ensured.

The effect of this special dimensioning of the screen 130 is that when the cavity examination device 100 is inserted into the cavity 140, as represented in FIG. 1a, the screen edge on the second opening is folded toward the body 110 of the device 100, i.e. away from the interior of the cavity 140, and when the movement is reversed, as represented in FIG. 1b, it is folded back and now folded away from the body 110 of the device 100, that is to say toward the interior of the cavity 140.

This firstly has the advantage that—unlike with the known funnel- or cone-shaped tips of U.S. Pat. No. 5,363,839—the optical reception element 120 of the body 110 is guided essentially in the middle of the cavity 140 to be examined and, owing to the use of the elastic material, this guiding cannot cause any injury to the wall and actually exerts only a minimal pressure on it.

Another advantage of the screen arrangement according to the invention will be explained below in connection with FIG. 2. The representation of FIG. 2 corresponds to that of FIG. 1b, and is supplemented by (flexible) foreign bodies 150, 155 which may be found in the cavity 140, for example hairs growing on the auditory canal wall. These are simply pressed aside by the screen, which is represented by displaced hairs 155, and therefore do not interfere with an image recording region 160 denoted by shading. Advantageously, these hairs do not elaborately need to be removed before the examination by shaving with special devices.

Advantageously, the parameters of the cavity examination device 100 are selected so that the screen cannot for its part impede the imaging. Selectable parameters may in particular be: aperture angle and/or focal length of the optical reception element 120; cross-sectional shape and cross-sectional configuration of the second opening 132 as well as the extent of the screen 130 in the direction from the first opening 131 to the second opening 132 (i.e. the height of the body of rotation frustum, which is preferably used).

In an exemplary embodiment, the screen 130 has a slit extending from the second opening, so as to be able to adapt better to varying cross sections of the cavity 140 to be examined.

In one configuration of the cavity examination device according to the invention for a medical application, for the examination of auditory canals, it is advantageous or even necessary to provide replaceable screens. On the one hand, after use, a device 100 can thereby be prepared rapidly for the next patient by fitting a fresh screen, and on the other hand it is possible to accommodate different sizes of auditory canal dimensions by using a correspondingly dimensioned screen.

The 3D measurement of an auditory canal, or more generally a cavity 140, with the cavity examination device 100 according to the invention, may be carried out by initially inserting the device as far as necessary into the cavity 140. The image recording is then started, and the device is removed again from the cavity with a suitable speed. The image sensor (not shown), either used directly as the reception element 120 or arranged behind a lens functioning as the reception element, then forwards the recorded images to an analysis device (not shown) which stores the image sequence and computes a 3D model of the cavity in real time or after a delay.

The optical measurement of the auditory canal is not interfered with by hairs and the like. The optical reception element 120 is always approximately centered, and the auditory canal wall only comes in contact with the soft silicone screen, but not with the hard body 110.

It should be pointed out that the present invention is not restricted to cavity examination devices which compile a 3D model of the cavity, but for example may also be used for (video) otoscopes or combined measurement and otoscopy examination devices.

Naturally, any other desired flexible material may also be used besides silicone as the flexible material, for example rubber, natural rubber or the like.

Besides rigid device bodies 110, it is furthermore conceivable to use flexible device bodies in connection with the present invention, for example in order to be able to examine curved channels.

1-10. (canceled)

11. A cavity examination device, comprising:
a body having a preferred cavity insertion direction and a forward end to be inserted first into the cavity;
an optical reception element for receiving light emitted or reflected from a wall of a cavity; and
a screen of flexible material formed with a first opening and a second opening;
said second opening having a cross-section greater than a cross-section of said first opening, and said second opening being defined by a configuration and dimension to predominantly bear resiliently on the wall of the cavity;
said screen being fastened to said body at said first opening at said forward end of said body of the cavity examination device to be inserted first into the cavity; and
wherein said screen is folded backward, relative to the cavity insertion direction, on said body when the cavity examination device is being inserted into the cavity and said screen is folded forward when the cavity examination device is being moved out of the cavity, with said screen folded away from said body when the cavity examination device is being retracted from the cavity.

12. The cavity examination device according to claim 11, wherein said screen is a silicone screen.

13. The cavity examination device according to claim 11, wherein said screen substantially has a shape of a frustum of a body of rotation.

14. The cavity examination device according to claim 11, wherein said screen is dimensioned so as not to interfere with an imaging by said optical reception element.

15. The cavity examination device according to claim 11, wherein said screen is interchangeably mounted for replacement by a screen having a differently configured and/or dimensioned second opening, in order to adapt the cavity examination device to different cavities.

16. The cavity examination device according to claim 11, wherein said screen is formed with one or more slits for use in tapering cavities.

17. The cavity examination device according to claim 11, wherein said body is a cylindrical body.

18. The cavity examination device according to claim 11, wherein said optical reception element is an image sensor, a camera, or a front lens of a camera lens configuration, and wherein said image sensor or said camera records an image sequence when the cavity examination device is being retracted from the cavity, and feeds the image sequence to a control device that computes a 3D model of the cavity from the image sequence.

19. The cavity examination device according to claim 11, wherein said body and said screen are dimensioned for examining a human or animal auditory canal.

20. The cavity examination device according to claim 11, wherein said screen is dimensioned so that, when the cavity examination device is moved out of the cavity, said screen keeps flexible foreign bodies present in the cavity away from an imaging region of said optical reception element.

21. The cavity examination device according to claim 20, wherein said body and said screen are dimensioned for examining a human or animal auditory canal, and said screen is dimensioned to keep hairs growing in the auditory canal out of the imaging region.

22. The cavity examination device according to claim 11, wherein said first opening is disposed in front of said second opening, in the insertion direction, as the cavity examination device is being inserted into the cavity and said second opening is located deeper in the cavity than said first opening as the as the cavity examination device is being retracted from the cavity.
